(19) United States
(12) Patent Application Publication
(10) Pub. No.: US 2002/0076434 A1
(43) Pub. Date: Jun. 20, 2002

(54) Liposomal retinoid therapy in hormone refractory prostate cancer

(75) Inventor: Anthony H. Williams, Cambridge, MA (US)

Correspondence Address: Thomas M. Saunders, Lorusso & Loud, 440 Commercial Street, Boston, MA, 02109, US

(73) Assignee: Aronex Pharmaceuticals, Inc.

(21) Appl. No.: 09/975,555

(22) Filed: Oct. 12, 2001

Related U.S. Application Data
(60) Provisional application No. 60/240,775, filed on Oct. 16, 2000.

(51) Int. Cl. A61K 9/127

(52) U.S. Cl. 424/450

(57) ABSTRACT

A method of inducing chronic remission of HRPC as well as progressive metastatic disease in a human comprising the method of administering liposomal ATRA at a dosage of at least about 100 mg/m² at least about 2 or 3 days per week for a treatment cycle of at least about 25 to about 35 days for at least about 5 or 6 treatment cycles.

### FIELD OF THE INVENTION

A method of inducing chronic remission of HRPC as well as progressive metastatic disease in a human comprising the method of administering liposomal ATRA at a dosage of at least about 100 mg/m² at least about 2 or 3 days per week for a treatment cycle of at least about 25 to about 35 days for at least about 5 or 6 treatment cycles.

### BACKGROUND OF THE INVENTION

Prostate cancer is a common cause of cancer death in the US. Treatment options are particularly important once hormone-refractory prostate cancer (HRPC) becomes resistant to hormonal suppression. In men, prostate cancer is the most common cancer diagnosed, and the second most common cause of cancer death in the United States. Despite advances in medicine, prostate cancer still poses an enormous healthcare problem and its treatment remains a complex issue. Without being bound by any particular theory, it is widely accepted that androgen deprivation therapy (ADT) is an inadequate first-line treatment in metastatic prostate cancer. ADT is not curative. The duration of ADT response is often short (median, 12 to 18 months), with almost all subjects succumbing to their disease. Treatment options, once the disease becomes resistant to hormonal suppression, include secondary hormonal therapy, and chemotherapy.

Vitamin A, which is essential for the maintenance of normal epithelial structure and function in the body, is a modulator of the growth and differentiation of prostatic epithelial cells. Retinoids, a class of chemical compounds structurally related to vitamin A, have shown activity in several types of cancers and have been shown to be an effective agent in inducing remission in patients with acute promyelocytic leukemia (APL). Liposomal ATRA, in particular, has been demonstrated to be safe and effective in inducing both hematologic and molecular complete remission (CR) in APL, especially in patients where the administration oral ATRA is not feasible.

Kelly et al. Report a phase II trial using oral ATRA (non-liposomal) at a dose of 50 mg/m² administered every 8 hours for 8 weeks in 14 HRPC patients with bidimensionally measurable prostate cancer. Trump et al. report a similar study in 17 HRPC patients with distant metastatic disease with oral ATRA given on days 1-14, repeated every 22 days. Both studies determined that there was little tumor response, despite dose modification in the latter study in an attempt to avoid the self-induced ATRA clearance mechanisms associated with the use of oral (non-liposomal) ATRA. Particular note is made of the fact that free (non-liposomal) form of all-trans retinoic acid are recognized as effective in the treatment of cancer but only for brief periods.

Culine et al. reports using oral ATRA at a dose of 45 mg/m² administered daily for 7 days followed by 7 days of no treatment and then resumed treatment on Day 14 in 26 HRPC patients with manifestations of progressive metastatic disease. This dose schedule was also an attempt to avoid the self-induced ATRA clearance mechanisms in order to provide adequate drug exposure. They report 4 biological responses in the group of patients studied. One of these 4 patients, who had a baseline prostate-specific antigen (PSA) value of 25.0 ng/mL at study entry, achieved CR as the serum PSA value returned to within the normal limits and remained within the normal limits for 8 weeks after the last dose but exhibited a 3- month maximum response produced by oral ATRA. This particular patient had asymptomatic, minimal bone disease at study entry. In summary, previous studies have failed to demonstrate chronic remission in the use of non-liposomal ATRA in HRPC patients with progressive metastatic disease.

Other aspects of ATRA and liposomal; ATRA are noted in U.S. Pat. No. 5,811,119, the teachings of which are incorporated herein by reference. Particular note is made of using the liposomal all-trans retinoic acid in one subject administered 60 mg/m² every other day for 15 doses i.v. The C_{bO} (the concentration in blood at the conclusion of i.v. administration, time 0) in μg/ml was 6.8 on day one and 7.0 on day 15 after the eighth dose. The AUC in μg/ml×min was 466 on day 1 and 580 on day 15. Converting to μg hr/ml these values are 7.76 and 9.66 respectively.

### SUMMARY OF THE INVENTION

The present invention includes a method of inducing chronic remission of HRPC in a human comprising the method of administering liposomal ATRA at a dosage of at least about 100 mg/m² at least about 2 days per week for a treatment cycle of at least about 25 days for at least about 5 treatment cycles. In particular embodiments dosages are at least about 100 mg/m², at least about 120 mg/m², at least about 140 mg/m², and at least about 160 mg/m². Dosages as high as about 300 mg/m² are contemplated. In instances where toxicity is a clinical problem, dosages are usefully reduced in about 10 mg/m² to about 30 mg/m² increments and particularly about 20 mg/m² increments until acceptable levels of adverse effects are reached. It is contemplated that administration of liposomal ATRA will be from about daily to about once per week, as in maintenance applications. Particular note is made of dosing about 2 times per week and about 3 times per week. Dosing cycles are conveniently measured in 4 week or about 28 day cycles. In practice, cycles are usefully from about 25 to about 25 days.

L-ATRA is introduced parenterally with particular reference to intravenous administration. In intravenous administration, introduction over a period of about 20 to about 40 minutes is noted with particular reference to about 30 minutes.

Particular embodiments of the method are applicable to early stage HRPC, to HRPC subjects naive to chemotherapy and to treating humans subjects with HRPC and progressive metastatic disease. Reference is made to Stage 1 prostate cancer or stage A in the Whitmore-Jewett staging system.

A particular embodiment of the invention includes a method of inducing chronic remission of HRPC and particularly early stage HRPC in a human comprising the method of administering liposomal ATRA at a dosage of at least about 2.4 at least about 2 days per week for a treatment cycle of at least about 25 days for at least about 5 treatment cycles, and optionally administering liposomal ATRA at a dosage of at least about 3.4 mg/kg/day at least about 3 days per week for a treatment cycle of at least about 28 days for at least about 6 treatment cycles. In specific applications of this method L-ATRA is administered intravenously, including over a period of at least about 20 minutes, and optionally over a period of about 30 minutes or longer. In this method, reference is made to Stage 1 prostate cancer or stage A in the Whitmore-Jewett staging system.

This method is particularly contemplated for human subjects naive to chemotherapy. This method is also contemplated for treating humans subjects with HRPC and progressive metastatic disease comprising intravenously administering at least about 3 days out of seven at least about 2.4 mg/kg/day of liposomal ATRA, and particularly comprising administering daily at least about 3.4 mg/kg/day.

Applicants invention is drawn to methods of ATRA or retinoid therapy which avoids retinoic acid resistance. Liposomal retinoids do not give rise to retinoic acid resistance in chronic use and that this is the solution to a long-felt need.

### DETAILED DESCRIPTION OF THE INVENTION

An improved treatment is now presented. Intravenous liposomal all-trans-retinoic acid (ATRA) is effective in treating HRPC.

This invention will be better understood with reference to the following definitions:

A. “Chronic remission as to HRPC shall be understood to mean remission in excess of about 3 months, with particular reference to about 4 months, six months or more and including about 11 months or more.

B. “HRPC” shall mean,hormone refractory prostate cancer. HRPC is resistant to forms of treatment based on the use of hormones. In this context, “refractory” shall be understood to mean a disease state that fails to respond to doses of appropriate drugs that are effective in most patients (falling generally between the minimum inhibitory concentration (MTD) and maximum tolerated dose (MTD), or doses that were previously effective in that patient. “Relapsed” shall be understood to mean the presence of a rising PSA or the appearance of new metastases in individuals previously in complete remission.

Based on these definitions, it will be appreciated that an oncology medical professional evaluating whether or not a subject has relapsed or refractory cancer shall mean comparing a subjects presenting condition with the standards of relapsed and refractory as defined above. It is understood that clinical presentation is accompanied by a degree of variability, but the evaluation of subjects as relapsed or refractory is within the skill of an oncology medical professional.

C. “Early stage” in reference to HRPC shall mean a disease which has not yet been treated extensively with cytotoxic chemotherapeutic agents.

Early stage also includes Stage 1 prostate cancer defined as a cancer that is only in the prostate gland, cannot be felt during a digital rectal examination, is not visible by imaging, and causes no symptoms. It is usually found accidentally or because a blood test showed an elevated prostate-specific antigen level. Cancer cells may be found in only one area of the prostate or they may be found in many areas of the prostate. Similar to stage A in the Whitmore-Jewett staging system.

D. “Naive to chemotherapy” as to a subject shall mean that the subject has not received the cytotoxic chemotherapeutic agents. By way of nonlimiting example, reference is made to such cytotoxic chemotherapeutic agents including alkylating agents (nitrogen mustards, ethylenimines, alkyl sulfonates, nitrosoureas and triazines), antimetabolites (folic acid analogs, pyrimidine analogs, and purine analogs), vinca alkaloids, epipodophylotoxins, antibiotics (actinomycin D, Bleomycin, plicamycin, doxorubicin, daunorubicin, and mitomycin C), enzymes such as I-asparaginase, cis-platinum and related compounds of broad antineoplastic activity on proliferating cells. Monoclonal antibodies are not of general cytotoxic activity and those directed to specific cell surface recognition sites are not considered cytotoxic chemotherapeutic agents. However, some monoclonal antibodies such as Mylotarg (University of Maryland Greenebaum Cancer Center, Md.), have very specific marrow suppressive activity, and therefore are considerd cytotoxic chemotherapeutic agents in the present instance.

E. “Progressive metastatic disease” shall mean cancer that is increasing in scope or severity at multiple locations with a subject. In this context, progressive shall be understood to include both the appearance of new lesions, and/or a rising PSA.

F. ATRA refers to all trans retinoic acid, a retinoid. Retinoids in general include trans-retinoic acid and all-trans-retinol. Other retinoids are retinoic acid methyl ester, retinoic acid ethyl ester, phenyl analog of retinoic acid, etretinate, retinol, retinyl acetate, retinaldehyde, all-trans-retinoic acid, and 13-cis-retinoic acid.

G. Liposomal ATRA or retinoid shall be broadly understood to encompass all lipid associated ATRA or retinoid forms. More narrowly defined, “liposomes” are generally spherical structures comprising lipids, fatty acids, lipid bilayer type structures, unilamellar vesicles and amorphous lipid vesicles. Classically, liposomes are completely closed lipid bilayer membranes containing an entrapped aqueous volume. Liposomes may be unilamellar vesicles (possessing a single bilayer membrane) or multilamellar vesicles (onion-like structures characterized by multiple membrane bilayers, each separated from the next by an aqueous layer). The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “head” orient towards the aqueous phase. Liposomes are vesicles composed of one or more concentric phospholipid bilayers and used medically especially to deliver a drug into the body. As used herein, and for convenience, drug:lipid aggregates will be included within the terms liposome and liposomal. By way of example of such nonliposomal lipid bearing forms, reference is made to U.S. Pat. No. 4,610,868 to Fountain, the teachings of which are incorporated herein by reference.

Reference is made to liposomal-ATRA and retinoids as disclosed in U.S. Pat. No. 5,811,119 “Formulation and Use of Carotenoids in the Treatment of Cancer the teachings of which are incorporated herein by reference.

For convenience, the term “liposomal-ATRA or -retinoid” shall extend to high ratio drug:lipid complexes that are no classically liposomes.

I. Therapeutically effective amount as to a drug dosage, shall mean that dosage that provides the specific chronic pharmacological response for which the drug is administered in some members of a population of subjects in need of such treatment.

In particular embodiments dosages are at least about 100 mg/m², at least about 120 mg/m², at least about 140 mg/m², and at least about 160 mg/m². Dosages as high as about 300 mg/m² are contemplated. In instances where toxicity is a clinical problem, dosages are usefully reduced in about 10 mg/m² to about 30 mg/m²increments and particularly about 20 mg/m²increments until acceptable levels of adverse effects are reached.

It is emphasized that cancer resistant to drug therapy and/or resistance which changes over the course of the disease is a known problem in the art. Reference to “specific pharmacological response for which the drug is administered in a significant number of subjects in need of such treatment” is a recognition that a “therapeutically effective amount,” administered to a particular subject in a particular instance will not universally “cure” or reduce the severity of the disease in a particular subject, even though such dosage is deemed a “therapeutically effective amount” by those skilled in the art.

J. “Treatment cycle” is a term to be broadly construed to address blocks of time allotted to a treatment regimen. The usual cycle is a four week cycle of weekly treatment being about 28 days. It will be understood that hospital and patient scheduling and convenience permits latitude in specific cycles from about 25 to about 35 days.

Testing of intravenous liposomal all-trans-retinoic acid (ATRA) in treating HRPC has included 26 human subjects. All 26 enrolled subjects had received prior hormonal treatment; 13 had received prior chemotherapy and/or radiation therapy. Liposomal ATRA (140 mg/m²) was administered thrice weekly for a maximum of 72 doses or until progressive disease was observed, as occurred in 21 subjects (80.8%). Two subjects (7.7%) were discontinued for treatment-related toxicities; 2 subjects (7.7%) completed the study. As with other retinoids, headache and dry skin were the most commonly reported adverse events. Biological response of decreased prostate specific antigen (PSA]) was observed in 3 of the 24 evaluable subjects (12.5%) in a group of subjects with early stage disease. This demonstrates that liposomal ATRA is useful for the treatment of HRPC with particular note as to the treatment of early-stage HRPC.

A. Materials and Methods

Subject Eligibility: A total of 26 subjects were enrolled in the study protocol. All subjects had histologically confirmed adenocarcinoma of the prostate with manifestations of progressing regional or metastatic disease despite primary hormonal therapy (bilateral orchiectomy, estrogen or luteinizing hormone-releasing hormone [LHRH] therapy). Subjects may have had one or more prior hormonal therapies, chemotherapy, or radiation therapy to the prostate and/or to metastatic site(s). Other eligibility criteria included a Zubrod performance status of ≧2 and an expected survival of ≧8 weeks. In addition, subjects were to have evidence of measurable or evaluable disease; if evaluable disease consisted only of elevated prostate specific antigen (PSA), PSA must be >10 ng/mL and documented to be rising on 2 separate occasions, 2 or more weeks apart. Exclusion criteria included radiation therapy or chemotherapy 14 days and 21 days prior to study entry, respectively; metastatic involvement of the central nervous system; known hypersensitivity to retinoid or to retinoic acid derivatives; history of second previous malignancy within the past 5 years (with the exception of non-melanomatous skin cancer); and experimental therapy in the past 30 days. Subjects with known congestive heart failure, unstable arrhythmia, or myocardial infarction were also excluded.

Pretreatment evaluation included a complete medical history, physical examination and vital signs, blood count, serum chemistry studies including lipid profile, serum PSA, chest x-ray, electrocardiogram, and radionuclide bone scan. Other studies including computerized tomography could be performed at the discretion of the Investigator. In addition, the subject was asked to complete Quality of Life (QoL) and Visual Analog Scales (VAS) as part of the pretreatment evaluation. Prior to drug administration on Day 1 of each 28-day treatment cycle, and at the end of the study, clinical examination, blood count, serum chemistry panels, Zubrod performance status, and completion of QoL and VAS were conducted. Measurable and evaluable disease assessments were repeated at the end of every 3 treatment cycles.

Treatment. The study drug used was liposomal-ATRA (ATRAGEN®, Aronex Pharmaceuticals, Inc., The Woodlands, Tex.), an intravenous liposomal formulation of all-trans-retinoic acid, supplied as 100 mg per 100 cc vial. The starting dose was 140 mg/m² administered intravenously over 30 minutes on Monday, Wednesday, and Friday of every week. This treatment schedule was continued until progressive disease (PD) occurred or until the completion of 6 treatment cycles (total of 24 weeks, maximum of 72 doses). If a limiting toxicity occurred, the was reduced in increments of 20 mg/m² depending upon the toxicity level experienced by the subject as defined by the World Health Organization (WHO) criteria. Subjects receiving therapy with LHRH analogues continued such treatment, but all other hormonal treatments were discontinued at least 4 weeks prior to study entry.

Response Assessment. Response was primarily assessed by serial measurements of serum PSA every 28 days. Biological response (BR) was defined as decrease of >50% in PSA value as compared to the baseline PSA value. Complete response (CR) was defined as normalization of PSA values (PSA value of <4.0 ng/mL) and partial response (PR) as 80% reduction in PSA values from baseline. Both responses were to be documented for 2 consecutive treatment cycles or more (total of 8 weeks or more). Progressive disease was defined as >100% increase in PSA values over the initial values sustained on 2 consecutive measurements obtained 2 or more weeks apart, a 25% increase in the sum of the perpendicular diameters of all measurable masses, malignant hepatomegaly, the appearance of new lesions, or whenever radiation therapy was required for pain control or for any other indications as determined by the Investigator.

Pharmacokinetics: Two subjects consented to participate in the pharmacokinetic study portion of the protocol. Plasma samples were obtained for ATRA analysis after the first, second, third, and fourth doses of the study drug during the first and third treatment cycles.

B. Results

Subjects. Subjects were aged 57 to 86 years (median of 74 years); there were 8 Hispanics, 6 blacks, and 12 Caucasians. Of the 26 subjects enrolled, 13 subjects (50%) had received 1 or more prior hormonal treatments alone, and the other 50% had received a combination of hormonal therapy, chemotherapy or radiation therapy to the prostate and/or to metastatic site(s). Seven subjects (26.9%) had bilateral orchiectomy prior to study entry. All except 4 subjects (14.8%) had some form of skeletal metastases at study entry.

Treatment delivery and toxicity. Six hundred and ninety five (695) doses of liposomal ATRA (median of 28 doses per subject, range of 9 to 67 doses) were administered for a total of 245 weeks (median of 11 weeks per subject, range of 4 to 24 weeks). Therapy was discontinued in 21 subjects (80.8%) because of PD (17 for increasing PSA levels, 2 for an increase in the sum of the perpendicular diameters of all measurable masses, and 2 needed radiation therapy for pain control); 2 subjects (7.7%) were discontinued for toxicities that were assessed to be probably related to the study drug; 1 subject (3.8%) was discontinued from the study due to the Investigator's judgment; and, 2 subjects (7.7%) completed the study.

The most common adverse events (AEs) reported were headache and dry skin, which were also reported to be most common among subjects with acute promyelocytic leukemia receiving liposomal ATRA. These AEs are common with oral ATRA and retinoids in general.

Responses. Of the 24 evaluable subjects, BR was observed in 3 subjects (12.5%). Of these 3 subjects, 2 subjects (8.3%) completed the study protocol. Of the 2 subjects who completed the study protocol, 1 subject had a PSA value of 154 ng/mL at baseline that decreased to 3.9 ng/mL after the fourth treatment cycle. PSA values were 0.05 ng/mL, 1.5 ng/mL, and 8.0 ng/mL at 1, 8, and 11.5 months, respectively. The other subject, who had bilateral orchiectomy, started with a PSA value of 123 ng/mL that decreased to 68 ng/mL at the end of the second treatment cycle and decreased further to 34 ng/mL 1 month following the last dose of the liposomal ATRA. Both of the subjects who completed the study had not received any previous radiotherapy or chemotherapy and had negative bone scan findings (no metastases) at study entry. These 2 subjects had generally had less prior therapy than other subjects. One subject with a history of failed leuprolide and estramustine therapies, (no prior radiation therapy, and negative bone scan findings at study entry) had stable disease despite a rising PSA. His PSA value was 166 ng/mL at the start of therapy and remained within 30% of this value during treatment (181 ng/mL, 209 ng/mL, and 186 ng/mL after the first, second, and third treatment cycles, respectively). One subject with a long-standing history of asthma was taken off the study protocol after 4 doses because of probable exacerbation of his condition by the study drug; this subject was considered nonevaluable for this study's efficacy analysis. In this particular subject, the study drug was reduced to 100 mg/m² on the third dose, after the subject complained of intermittent shortness of breath relieved by his current medications. Despite this dose reduction, the subject continued to experience the same symptom after every administration of the study drug. A chest x-ray was obtained and congestive heart failure was ruled out. This subject had a history of hormonal treatment with no history of previous radiotherapy or chemotherapy, and no evidence of skeletal metastases at study entry. His baseline PSA value of 102.0 ng/mL decreased to 87.5 ng/mL within 16 days of the first dose of the study drug.

There were 2 other cases of dose reductions from 140 mg/m² to 120 mg/m² of the study drug in 2 separate subjects enrolled on the study protocol. One subject's dose was decreased because of headache and the other subject's dose because of muscle weakness; both these events resolved after dose reductions were made.

One subject, who was on concomitant coumadin therapy, was taken off the study protocol after 2 doses of the study drug because of the probable contribution of the study drug to his increased International Normalized Ratio (INR) levels; he was also considered nonevaluable for purposes of efficacy analysis for this study. This subject, with a history of prior hormonal treatments and the presence of extensive bone metastases at study entry, had a baseline PSA value of 2012 ng/mL that increased to 3205 ng/mL within 35 days following the first dose of the study drug.

Pharmacokinetics. ATRA clearance in HRPC subjects was found to have a mean of 80.1±11.9 mL/min/m² (range, 70.3 to 93.3) which was lower when compared to the ATRA clearance in 11 healthy volunteers (mean of 98.5±20.41 mL/min/m²) who received 90 mg/m² of the study drug every other day for 15 days. A longer elimination half-life was found in HRPC subjects, mean of 1.94 ±0.190 hr and a range of 1.81 to 2.16, when compared to healthy volunteers (mean of 1.06±0.233 hr). ATRA volume of distribution was found to have a mean of 10.7 ±1.86 L/m² (range, 8.6 to 12.1) among HRPC subjects compared to a mean of 11.2±2.76 L/m² among healthy volunteers.

Liposomal ATRA-treated established a durable decrease in PSA, with a decrease to within normal limits after 4 cycles of therapy; This value remained within normal limits for the last 2 cycles of therapy and for 11.5 months following the last dose of liposomal ATRA (a total of 13.5 months). This is surprisingly superior to a 3-month maximum response produced by oral ATRA as documented by Culine et al.

Liposomal ATRA of the present invention has been found to result in greater exposure to ATRA for a longer period of time when compared to oral ATRA. As liposomal ATRA is unaffected by the self-induced ATRA clearance mechanisms that may cause oral ATRA's failure to provide adequate drug exposure, liposomal ATRA is an ideal drug to consider in prostate cancer as well as in other tumors sensitive in vitro to ATRA. In a specific embodiment liposomal ATRA's is of particular efficacy in the early stages of HRPC and that it will be a safe and well tolerated regimen to consider once the disease progresses to become resistant to hormonal suppression, but prior to initiating chemotherapy.

Reference is made to the following publications the teachings of which are incorporated herein by reference:

1. Lo Coco F, Dinerio D, Fabini B, et al.: Genetic Diagnosis and Molecular Monitoring in the Management of Acute Promyelocytic Leukemia. Blood 1999; 94: 12-22.

2. Sanz M, Lo Coco F, Marti G, et al.: Definition of relapse risk and role of nonanthracycline drugs for consolidation in patients with acute promyelocytic leukemia: A joint study of two PETHEMA and GIMEMA cooperative groups. Blood 2000, 96:1247-53.

3. Mandeli F, Dinerio D, Avvisati G, et al.: Molecular remission in APL/RAR alpha-positive acute promyelocytic leukemia by combined all-trans-retinoic acid and idarubicin (AIDA) therapy. Blood 1997; 90:1014-1021.

4. Sanz M, Martin G, Rayon C, et al.: A modified AIDA protocol with anthracycline-based consolidation results in high antileukemic efficacy and reduced toxicity in newly diagnosed PML/RAR alfa-positive acute promyelocytic leukemia. Blood 1999; 94:3015-21.

5. Estey E, Giles F, Kantarjian H, et al.: Molecular remission induced by liposomal-encapsulated all-trans-retinoic acid in newly diagnosed acute promyelocytic leukemia. Blood 1999; 94:2230-2235.

6. Fenaux P, Chastang C, Sanz M, et al.: A randomized comparison of ATRA followed by chemotherapy and ATRA plus chemotherapy and the role of maintenance therapy in newly diagnosed acute promyelocytic leukemia. Blood 1999, 94:1192-1200.

7. Tallman M, Andersen J, Schiffer C, et al.: All-trans-retinoic acid in acute promyelocytic leukemia. N Engl J Med 1997;337:1201-1208.

The compositions of this invention possess valuable pharmacological properties. They inhibit the proliferation of neoplastic cells while including those associated with Hormone Refractory Prostate Cancer as well as with other prostate cancers.

Thus, these compositions can be used in other cancers including blood cancers such as leukemia with particular reference to AML. Note is made of Hodgkin's disease, non-Hodgkin's lymphomas, acute and chronic lymphocytic leukemias, multiple myeloma, neuroblastoma, breast, ovary, and lung cancer, Wilms tumor cervix, testis, and soft tissue sarcomas. Further noted is chronic granulocytic leukemia, malignant melanoma, choriocarcinoma, mycosis fungoides, osteogenic sarcoma, hairy cell leukemia, Kaposi's sarcoma, essential thrombocytosis, and renal cancer.

Administration is contemplated to include chronic, acute or intermittent regimens. The compositions are particularly useful in the treatment of hormone refractory cancers such as HRPC.

Compounds of the present invention are also useful in screening procedures. They can be employed in admixture or co-therapy with other drugs. Particular reference is made to such anti-cancer drugs as alkylating agents (nitrogen mustards, ethylenimines, alkyl sulfonates, nitrosoureas and triazines), antimetabolites (folic acid analogs, pyrimidine analogs, and purine analogs), vinca alkaloids, epipodophylotoxins, antibiotics (actinomycin D, Bleomycin, plicamycin, doxorubicin, daunorubicin, and mitomycin C), enzymes such as I-asparaginase. Further note is made of biological response modifiers such as interferon-alpha, platinum coordination complexes such as cisplatin and carboplatin with specific reference to liposomal-cisplatin including diaminocyclohexane platinum complexes. Additionally noted are hormones and antagonists such as adrenocorticosteroids, progestins, estrogens, antiestrogen, androgens, antiandrogen, and gonadotropin releasing hormone analog (leuprolide). Specific mention is made of retinoids such as all-trans retinoic acid (ATRA) and liposomal ATRA, as well as 13-cis retinoic acid. In some embodiments, all-trans retinoic acid is administered at a dose of about 15 to 300 or more mg/m², and particularly at least about 90 mg/m^{2,} and at least about 140 mg/m².

The compositions of this invention are generally administered to animals, including but not limited to humans.

The pharmacologically active compositions of this invention can be processed in accordance with conventional methods of Galenic pharmacy to produce medicinal agents for administration to patients, e.g., mammals including humans.

The compositions of this invention can be employed in admixture with conventional excipients, i.e., pharmaceutically acceptable organic or inorganic carrier substances suitable for parenteral, enteral (e.g., oral or inhalation) application which do not deleteriously react with the active compositions. Suitable pharmaceutically acceptable carriers include, but are not limited to water, and salt or sugar solutions, The pharmaceutical preparations can be sterilized and if desired mixed with auxiliary agents, e.g., preservatives, stabilizers, emulsifiers, salts for influencing osmotic pressure, buffers and the like which do not deleteriously react with the active compositions. They can also be combined where desired with other active agents as noted above.

In some embodiments of the present invention, dosage forms include instructions for the use of such compositions.

For parenteral application, particularly suitable are injectable, sterile solutions, such as aqueous solutions and suspensions. Ampules are convenient unit dosages.

Sustained or directed release compositions can be formulated, e.g., liposomes or those wherein the active component is protected with differentially degradable coatings, e.g., by microencapsulation, multiple coatings, etc. It is also possible to freeze-dry the new compositions and use the lyophilizates obtained, for example, for the preparation of products for injection.

Generally, the compositions of this invention are dispensed in unit dosage form comprising about 150 to 500 mg of active ingredient in a pharmaceutically acceptable carrier per unit dosage.

The dosage of the compositions according to this invention generally are about 2.4 to about 7.3 mg/kg/day, particularly about 2.9 to about 3.9 mg/kg/day, and more particularly about 3.4 mg/kg/day when administered to patients, e.g., humans to treat HRPC.

It will be appreciated that the actual preferred amounts of active compositions in a specific case will vary according to the specific compositions being utilized, the particular compositions formulated, the mode of application, and the particular situs and organism being treated. Dosages for a given host can be determined using conventional considerations, e.g., by customary comparison of the differential activities of the subject compositions and of a known agent, e.g., by means of an appropriate, conventional pharmacological protocol.

1. A method of inducing chronic remission of HRPC in a human comprising the method of administering liposomal ATRA at a dosage of at least about 100 mg/m² at least about 2 days per week for a treatment cycle of at least about 25 days for at least about 5 treatment cycles.

2. The method of claim 1 wherein administering liposomal ATRA is at a dosage of at least about 140 mg/m² at least about 3 days per week for a treatment cycle of at least about 28 days for at least about 6 treatment cycles.

3. The method of claim 1 wherein L-ATRA is administered intravenously,

4. The method of claim 3 wherein the period of administration is over a period of at least about 20 minutes.

5. The method of claim 1 wherein HRPC is early stage HRPC

6. The method of claim 1 wherein said human is naive to chemotherapy.

7. A method of treating humans subjects with HRPC and progressive metastatic disease comprising intravenously administering at least about 3 days out of seven at least about 100 mg/m² of liposomal ATRA

8. The method of claim 7 further comprising administering daily at least about 140 mg/m² of liposomal ATRA.

9. A method of inducing chronic remission of HRPC in a human comprising the method of administering liposomal ATRA at a dosage of at least about 2.4 at least about 2 days per week for a treatment cycle of at least about 25 days for at least about 5 treatment cycles.

10. The method of claim 9 wherein administering liposomal ATRA is at a dosage of at least about 3.4 mg/kg/day at least about 3 days per week for a treatment cycle of at least about 28 days for at least about 6 treatment cycles.

11. The method of claim 9 wherein L-ATRA is administered intravenously,

12. The method of claim 11 wherein the period of administration is over a period of at least about 20 minutes.

13. The method of claim 9 wherein HRPC is early stage HRPC

14. The method of claim 9 wherein said human is naive to chemotherapy.

15. A method of treating humans subjects with HRPC and progressive metastatic disease comprising intravenously administering at least about 3 days out of seven at least about 2.4 mg/kg/day of liposomal ATRA

16. The method of claim 15 further comprising administering daily at least about 3.4 mg/kg/day of liposomal ATRA.
